(19) United States
(12) Patent Application Publication
(10) Pub. No.: US 2020/0289047 A1
(43) Pub. Date: Sep. 17, 2020

(54) FETAL MONITORING SYSTEM AND METHOD

(71) Applicant: KONINKLIJKE PHILIPS N.V., EINDHOVEN (NL)

(72) Inventors: YU QI, SHANGHAI (CN); CONG TIAN, SHANHAI (CN); YU QIANG WU, SHANGHAI (CN); ZHONGCHI LUO, SHANGHAI (CN); GUANGLI WEN, SHANGHAI (CN); BIN YIN, SHANGHAI (CN); LIN LI, SHANGHAI (CN)

(21) Appl. No.: 16/086,061

(22) PCT Filed: Mar. 21, 2017
(86) PCT No.: PCT/EP2017/056722

§ 371 Date: Sep. 18, 2018

(30) Foreign Application Priority Data
Mar. 21, 2016 (CN) PCT/CN2016/076872
May 10, 2016 (EP) 16168873.4

(51) Int. Cl. A61B 5/00; A61B 5/1455; A61B 5/11; A61B 5/1464

(52) U.S. Cl. CPC A61B 5/4362; A61B 5/14552; A61B 5/11; A61B 5/6831; A61B 2560/0475; A61B 5/0011; A61B 5/7278; A61B 5/1464; A61B 5/6823

(57) ABSTRACT

The invention provides a fetal movement monitoring system which uses optical pattern sensing to detect fetal movements. Fetal movements provide a change in the optical path between the optical sensor and detector, such as a different proportion of amniotic fluid and fetus and/or a different contact pressure with the optical sensor arrangements. One or both of these effects may be detected based on analysis of the optical signals captured by the system.

### FIELD OF THE INVENTION

This invention relates to a fetal movement monitoring system and method.

### BACKGROUND OF THE INVENTION

Fetal movement is of great importance for evaluating fetal health and wellbeing. Intact neuromuscular functions and an adequate supply of oxygen and nutrients to the central nervous system are needed for normal fetal movement. Decreases in fetal movement may indicate fetal compromise or they may predict poor fetal outcome. Pregnant women are suggested by healthcare providers to count fetal movement at least once every day, starting from the 2nd trimester. An average of 3 to 5 times per hour indicates the fetus is doing well. Too few or too many movements can be caused by a shortage of oxygen supply, which requires healthcare providers to be contacted.

Besides the clinical indications, information about the fetal movement (and position) may provide expectant parents with a general indication of how the fetus is doing, which gives pleasure and reassurance. This fetal diary or actigraph type of fetal activity recording, without explicit clinical implication, serves as an emotional link between the mother-to-be and the fetus.

Automation in recording fetal movements would be of great benefit. It is unrealistic to perform manual counting of fetal movements over a prolonged time period. For example, it may be desirable to record fetal movements over a period of an hour, and then to compare the results with a previous time period.

Known fetal movement monitoring systems make use of ultrasound Doppler sensing, accelerometers, piezoelectric pressure sensors and moving coil sensors. However, these techniques each have their own drawbacks. Ultrasound Doppler sensing involves exposure to radiation. Accelerometers are sensitive to all motion including those from the expectant mother. Piezoelectric pressure sensors for fetal movement sensing have relatively low detection sensitivity.

If a system is for home and continuous monitoring, ultrasound and galvanic technologies are not suitable, such as those that are adopted by most of the existing fetal monitoring products, either for medical or for consumer applications.

There is therefore a need for a simple, safe, easy to use and low cost system able to detect fetal movements. In particular, a wearable monitoring device would be of interest which is able to record and extract fetal movement information in an automatic fashion.

### SUMMARY OF THE INVENTION

The invention is defined by the claims.

According to examples in accordance with an aspect of the invention, there is provided a fetal movement monitoring system comprising:

an abdominal belt;

an optical sensor arrangement carried by the belt, comprising at least one light emitter for emitting an optical signal toward abdomen of an object and at least one light receiver for receiving the optical signal from the abdomen of the object and

a controller adapted to analyze optical signals received by the optical sensor arrangement thereby to detect fetal movements based on a pattern change of the optical sensor arrangement signals;

wherein the optical signal is directed to travel through the abdomen of the object.

This system makes use of optical monitoring to detect fetal movements. In particular, fetal movements provide (1) a change in the optical path between the optical sensor and detector, such as a different proportion of amniotic fluid and fetus, and/or (2) a different contact pressure with the optical sensor arrangements and the contact pressure change may lead to the change of a portion of the optical path, such as the arterioles of abdominal skin or/and muscles. One or both of these effects may be detected based on analysis of the optical signals captured by the system. In this way, a simple and low cost system is able to detect fetal movements, and provide a log over time of such movements.

The pattern change may be identified by temporal and/or frequency changes in the optical signals received.

The optical sensor may comprise a PPG sensor.

This provides a low cost and existing sensor technology. A PPG signal also enables the maternal respiration cycles to be detected, and these can be also be used in the analysis of the optical sensor arrangement signals to provide further improvement to the detection of fetal movements. For example, it is possible to compensate for movements of the expectant mother during respiration. Other physiological information can also be obtained, including the maternal and fetal heart rate, heart rate variability and oxygen saturation. A PPG sensor is highly sensitive and low cost, and also enables continuous monitoring of fetal movement.

The optical sensor may comprise a pulse oximetry sensor for implementing the PPG sensor.

Pulse oximetry measures the absorption of light and enables low power operation. With one choice of optical source (red or infrared) the signal penetrates into the fetus. The pulse oximetry may be then used to measure the fetal pulse at one frequency, but changes in the pulse oximetry signals at a lower frequency may be used to determine changes in the optical path resulting from fetus movement.

With a different choice of optical source (e.g. green light) the signal is reflected from the abdomen wall and instead may be used to measure the maternal pulse. Fetal movement, in particular a kick to the abdomen wall, changes the contact force, which leads to occlusion of the arterioles, and this changes the optical signal detected.

The system may further comprise a motion sensor.

A motion sensor may be used to enable optical signal changes which result from movement of the expectant mother to be cancelled, so as to enable more accurate determination of the optical signal changes resulting only from fetal movements.

The motion sensor may be carried by the belt. This makes the sensor part of the system, and therefore easy for the user to apply as a single belt.

The motion sensor may be mounted on the belt away from the fetal area. This ensures that the motion sensor is detecting movements of the expectant mother rather than the fetus.

The motion sensor, or a further motion sensor, may be worn by the expectant mother away from the belt.

The motion sensor may in this way be worn away from the abdomen, for example on the wrist. There may be motion sensors in multiple locations, and a wrist mounted device such as a smart watch may also function as the user interface for the system. The motion sensor may be carried, for example implemented by a mobile telephone.

The pattern of the optical signals received may be characterized by at least a time domain parameter or at least a frequency domain parameter or the combination of both. By performing time domain and/or frequency domain analysis of the optical signals, different types of fetal movement may be discriminated from each other. In this way, the system may provide a count of fetal movements within a particular time period and also an indication of the type of movement (such as kicking, or changing position).

The system may further comprise an output device, wherein the controller or the optical sensor arrangement communicates wirelessly with the output device.

The output device may be a smart watch, smart phone, laptop or other device on which the collected data may be displayed and analyzed. The data processing performed by the controller may be carried out on the belt (so the controller is at the belt) or at the output device (so the controller is at the output device), or some of the data processing may be carried out remotely. Similarly, the memory may be at the belt or at the output device.

The light emitter may be adapted to emit:

green light; or

red light or infrared radiation.

Note that the term “light” and “optical signal” when used generally is intended to cover all of the electromagnetic spectrum, and thus include visible light as well as infrared radiation for example.

Green light gives lower penetration depth, for example for a maternal PPG measurement and detection of pressure changes, whereas red light or infrared radiation gives a larger penetration depth for example for a fetal PPG measurement and detection of path changes.

Examples in accordance with another aspect of the invention provide a fetal movement monitoring method comprising:

generating an optical signal and receiving a reflected optical signal from a fetal area of an expectant mother using an optical sensor arrangement provided on an abdominal belt; and

analyzing the optical sensor arrangement signals thereby to detect fetal movements based on a pattern change of the optical sensor arrangement signals.

The method may comprise using a PPG sensor to generate and receive the optical signal. The method may further comprise using motion information relating to the expectant mother to assist in analyzing the optical sensor signals. Information about the fetal movements is preferably stored in a memory.

The optical signal may be generated with green light or red light or infrared radiation.

### BRIEF DESCRIPTION OF THE DRAWINGS

Examples of the invention will now be described in detail with reference to the accompanying drawings, in which:

FIG. 1 shows a fetal movement monitoring system;

FIG. 2 shows in schematic form the components of a PPG sensor such as a pulse oximetry sensor;

FIG. 3 shows the influence of contact pressure on a PPG signal;

FIG. 4 shows the belt of the system of FIG. 1 in more detail;

FIG. 5 shows the electrical/electronic system components for the system of FIG. 1;

FIG. 6 shows first experimental results for a first approach based on detecting contact pressure;

FIG. 7 shows second experimental results for the first approach based on detecting contact pressure;

FIG. 8 shows third experimental results for the first approach based on detecting contact pressure;

FIG. 9 shows the influence of fetal movement on the optical path between the light emitter and detector of a PPG sensor;

FIG. 10 shows how low pass filtering removes noise from the PPG sensor signal;

FIG. 11 shows first experimental results for a second approach based on detecting optical path changes;

FIG. 12 shows second experimental results for the second approach based on detecting optical path changes;

FIG. 13 shows third experimental results for the second approach based on detecting optical path changes;

FIG. 14 shows how different types of fetal movement produce different signal patterns in the time domain and in the frequency domain; and

FIG. 15 shows a general computer architecture suitable for performing the signal processing required by the system of FIG. 1.

### DETAILED DESCRIPTION OF THE EMBODIMENTS

The invention provides a fetal movement monitoring system which uses optical pattern sensing to detect fetal movements. Fetal movements provide a change in the optical path between the optical sensor and detector, such as a different proportion of amniotic fluid and fetus, and/or a different contact pressure with the optical sensor arrangements. One or both of these effects may be detected based on analysis of the optical signals captured by the system.

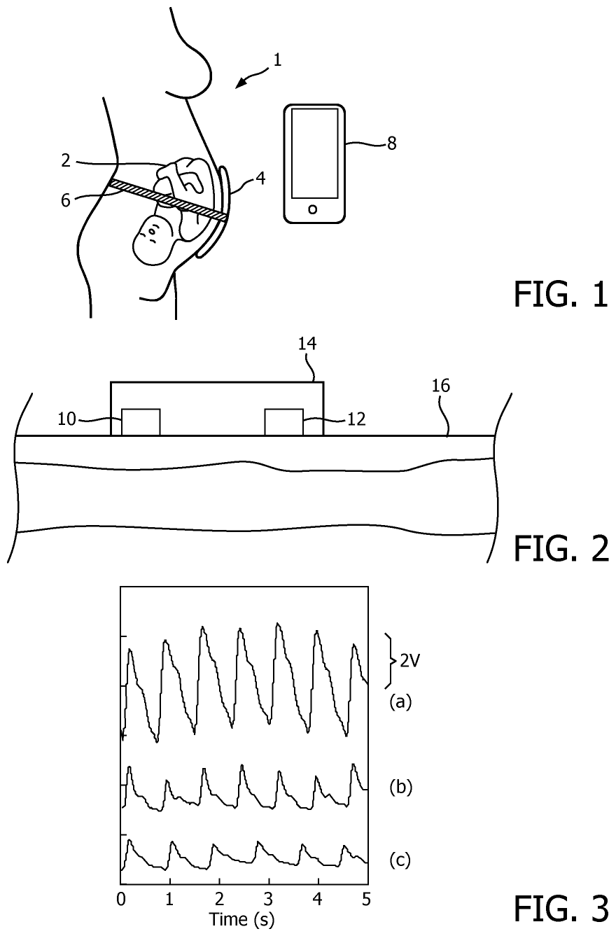

FIG. 1 shows one example of the system being worn by an expectant mother 1. The system is for monitoring movement of the fetus 2. An optical sensor array 4 is mounted over the belly comprising at least one light emitter and at least one light receiver, held on in this example by a strap 6. It may instead be temporarily adhered in place. The optical sensor array 4 and strap 6 together form a belt.

A processor is provided for processing the detected optical sensor signals thereby to detect fetal movements based on a pattern change of the optical signals received. These pattern changes are typically temporal changes in the optical sensor array signals but they may additionally be frequency spectrum changes. In the example shown, the processor is provided in a remote device such as a smart phone 8 (or smart watch) to which the sensor signals are transmitted from the sensor array 4 wirelessly. The processor may instead be part of the system, for example mounted on the belt. The data communication also may be over a wired connection to the processor rather than wireless. The processing may also be carried out remotely at a central back-end processing location, with communication for example over the Internet.

There is a memory for storing the sensor data, and for storing the results of the processing. In the example of FIG. 1, the smart phone 8 implements the memory and processor.

The invention makes use of optical sensing and pattern analysis. One example of suitable optical sensing technology is the photoplethysmogram (PPG). This is a known optical, non-invasive method used to detect volume changes in the blood vessels and most commonly used to measure the heart rate based on the changes in light absorption in the tissue at a specific wavelength.

The PPG method has the advantage of using low power levels that are harmless to test subjects. PPG is hence suitable for continuous monitoring; and it is also suitable for real time signal acquisition due to its fast response. The PPG system can be designed to be portable, robust and at low cost.

A reflectance-type PPG system is shown schematically in FIG. 2. There is an LED light emitter 10 and a photodetector 12 formed as part of a sensor probe 14 which is applied to a measurement site, such as the skin 16.

The received PPG signal waveform is affected by the contact force between the probe 14 and the measurement site 16 and thus differs depending on the PPG probe contact pressure. PPG signal recording under high pressure conditions can lead to low alternating current (AC) signal amplitude, as well as distorted waveforms caused by the occluded arterioles beneath the PPG probe 14.

A previous study has analyzed the pulse contour changes with increasing contacting force, and the results are repeated in FIG. 3. From FIGS. 3(a) to 3(c) the contact force is increased progressively, and the amplitude of PPG signal waveform decreases correspondingly. The contact forces are 0.21 N, 0.40 N and 0.81 N, and the results are repeated from Teng X F and Zhang Y T, “The effect of contacting force on photoplethysmographic signals”, Physiol. Meas. 2004; 25:1323-1335, which suggests to keep a constant force during PPG measurement to avoid signal distortion.

A first approach in accordance with an example of this invention makes use of this dependency of a PPG signal on the contact force between the measurement site and the probe. Fetal movement and kicking will generate pressure on the maternal abdomen. By detecting this generated pressure, fetal movements and kicks may be detected based on the interruption or reduction in intensity of the PPG signal due to the contact force changes between the measurement site and probe. The LED emitter 10 emits the green light towards the abdomen of the object. The green light directed to the abdomen can only penetrate the maternal tissues and then some of the green light propagates back to the light receiver 12 due to reflection, scattering or diffusion. When the force is generated due to the fetal kick or the fetal movement, the occlusion of arterioles happens or partially happens and then the composition of the propagation channel changes, which leads to the absorption change of the green light. Accordingly, pattern or intensity change of the received green light will take place at the detector 12 side.

FIG. 4 shows the sensor array 4 and strap 6. The sensor array comprises an array of PPG sensors 20, each having a light emitter and light detector (as shown in FIG. 2).

The sensors 20 of array 4 are mounted on a support 22, which is made of soft fabric and covers the whole abdomen in the front. It can be fixed on the back by the strap 6 with a Velcro connection, so that the overall system forms an adjustable belt.

The distance between the adjacent sensors 20 can either be fixed or adjustable. An arrangement with more sparsely distributed sensors in the array has a lower cost but lower sensitivity, whereas an arrangement with more densely populated sensors has a higher sensitivity but higher cost.

At the limit, there may be a single sensor (emitter and detector pair) but there is preferably an array or several or tens of sensors.

An accelerometer 24 (or accelerometer array) is provided as part of the monitoring system. In FIG. 4, it is shown on the strap 6, and therefore away from the abdomen. In this way, it does not detect localized movement of the belly area, but instead is used to monitor general trunk movement of the expectant mother.

The accelerometer may instead be a separate device which is worn or carried by the expectant mother away from the belt.

The system also has a controller for processing the sensor signals thereby to detect fetal movements based on a pattern change of the optical signals received. As explained with reference to FIG. 1, the controller may be part of a smart phone, or smart bracelet or watch. This controller device may for example also include the accelerometer.

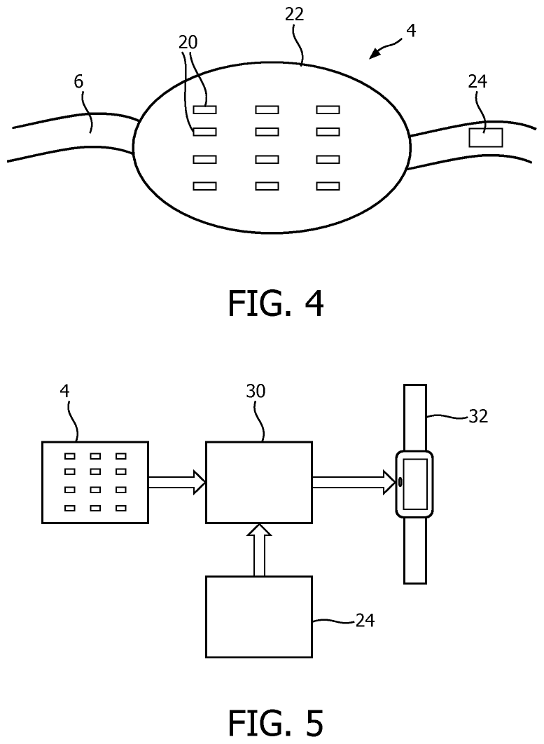

FIG. 5 shows the overall system electrical components. The sensor array 4 and the accelerometer 24 provide their outputs to the controller 30, which in this example is shown as separate to the output device, and the output device is a smart watch 32 which then functions simply as a display output device. The controller 30 includes a memory for storing historical data.

The controller 30 receives the PPG signals from the array of PPG sensors as well as the acceleration sensor signals in real time, and stores this information. An analysis module of the controller 30 analyzes the PPG waveform signals stored in the memory in order to identify fetal movements and to provide a movement count during a certain period, such as 1 hour. The analysis module of the controller 30 can also generate a fetal movement pattern over a longer period of monitoring, such as weeks and months, to inform the expectant mother about the wellbeing of the fetus. These results can be stored in a memory and displayed on the screen.

The accelerometer 24 is used to monitor maternal movements, which serves as a reference to reduce maternal motion artifacts. The possible influencing factors on the PPG signals include maternal motion, maternal respiration, displacement of the sensor relative to the body, and changes of ambient light, which can be eliminated by various means. The accelerometer can monitor the movement of the expectant mother to reduce the influence of the maternal motion artifact on the PPG signal. In particular, when the PPG signal variation overlaps in time with maternal movement sensed by the accelerometer, the PPG signal can be disregarded and thus not used to detect fetal movement. The influence of regular abdominal movement introduced by maternal respiration can also be removed by a signal processing algorithm.

Relative displacement of the PPG sensor relative to the measurement site can be effectively minimized during the counting of fetal movements by sitting or lying quietly during the monitoring period, and also by adjusting the length of the extendable belt to the correct length.

To eliminate the influence of invalid data collected during vigorous activity of the expectant mother, a signal processing algorithm can also be employed to analyze the PPG signals and accelerometer signals. When the maternal physical activity monitored by the accelerometer (or accelerometer array) is lower than a pre-determined threshold for a certain time period, the collected PPG data can be processed for analysis of fetal movement, while the signals are regarded as invalid when the physical activity of the expectant mother is higher than the threshold.

Changes of ambient light levels have minimal impact on PPG signals if the monitoring system is covered, for example by an outer housing and also by clothes of the expectant mother.

The controller 30 may also implement an alarm to alert the expectant mother when the fetal movement pattern changes as compared with previous stored normal movement patterns, or when the number of movements is too great or too low during a certain time period, such as 1 hour.

The PPG sensor array may also be used to measure the maternal blood perfusion signal which can be used to monitor maternal heart rate, heart rate variability and blood oxygen saturation.

In this first approach, which involves detection of contact force, the PPG light signal does not need to penetrate deep into the tissue, since a surface pressure effect is being monitored. Thus, the PPG sensors may use green light which only primarily penetrates into the mother's tissue, where the intensity change of the received optical signal mainly indicates the contact force change. The sensors may instead use red light or infrared radiation, with a deeper penetration depth, in which case the received optical signal is affected by influences beyond only the contact force. Other physiological information may then be obtained relating to the fetus.

Experiments have been conducted to demonstrate the effectiveness of the approach, by examining how PPG signals change in response to transient as well as continuous contact force changes.

In a first test, a PPG probe was attached to the finger of a subject and a force sensor was placed on top of the probe. The contact force between the finger and the PPG probe was changed by pressing down on the probe. Both continuous and transient pressure pulses were applied.

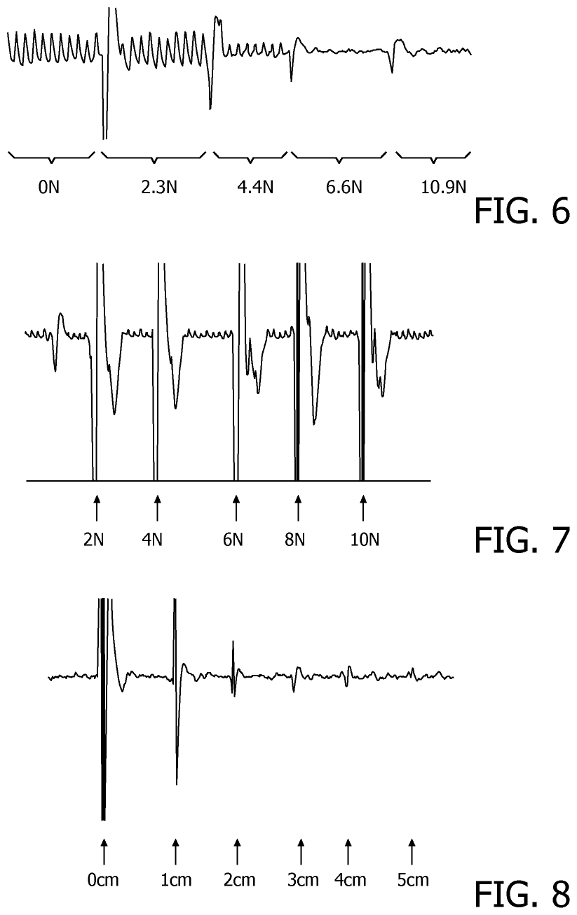

FIG. 6 shows the PPG signal for five different pressure levels (including zero pressure) versus time. Each new pressure level is applied as a continuous pressure for a period of approximately 10 seconds. The change in amplitude of the PPG signal at each different pressure level is clearly visible. When the pressure is high enough, the PPG signal disappears due to blocking of blood perfusion. This result indicates that the PPG waveform signal changes with different continuous contact force between the probe and measurement site, and such change persists with the continuous pressure. A more pronounced change in the optical signal pattern can also be observed at the time instants when there is a change in pressure.

FIG. 7 shows the PPG signal for five different pressure levels versus time. Each new pressure level is applied as a transient pressure pulse for a period of approximately 1 second. Each time a new pressure signal is applied, there is a significant disturbance to the PPG signal, regardless of the pressure level.

In a second test, a PPG probe was attached to the abdomen and a pressure was first applied on top of the probe, and then at successively further distances from the probe.

FIG. 8 shows the PPG signal versus time. The pressure was applied as a transient pressure pulse for a period of approximately 1 second. The pressure pulse is applied at the probe (shown as 0 cm) and then at successively further distances of 1 cm to 5 cm from the probe.

The results show that when a pressure pulse is applied on top of the probe, but also nearby the probe attached to the abdomen, the PPG waveform signal changes in response to the pressure, and the amplitude of the change reduces with the increased distance between the probe and the site where the pressure is applied. At distance of 5 cm, the change of PPG waveform signal is almost non-detectable. This result shows that pressure applied not only directly on top of the probe but also nearby the probe can cause the PPG signal waveform to change.

The approach above is based on using PPG (or other optical sensing) as a pressure detection mechanism.

A second approach is based on using the effect of changes in the optical path between the optical source and detector as a detection mechanism.

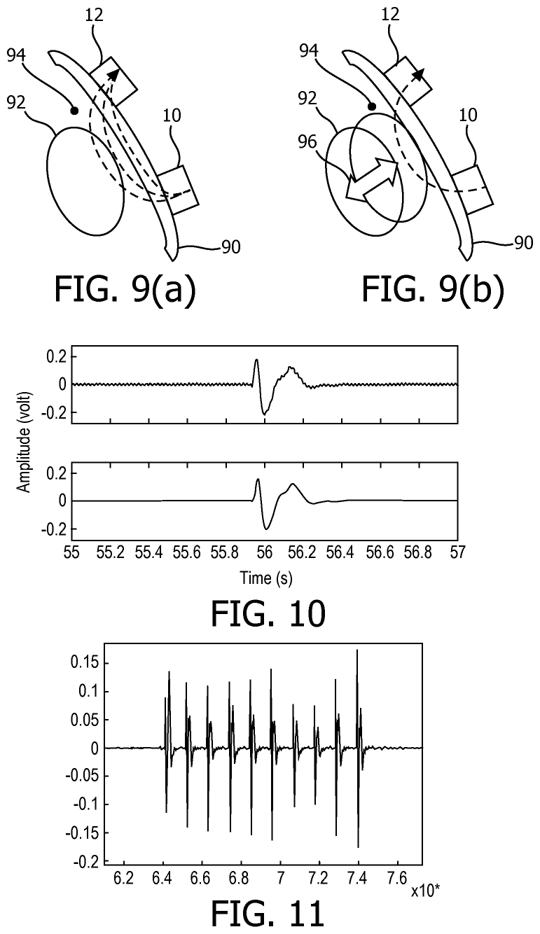

FIG. 9(a) shows that there is a set of optical paths between the source 10 and detector 12. The optical light is emitted by the LED transmitter 10 and directed to the abdomen of the object towards the fetal area through the multiple propagation paths. The light received due to reflection, scattering or diffusion at the detector 12 comes from a combination of paths. Some of the incident light has traveled through maternal tissue 90 as well as the fetal tissue 92 (and the amniotic fluid 94 between), some of the light has traveled only through the maternal abdominal tissue 90, and some has travelled through the maternal abdominal tissue 90 and the amniotic fluid 94.

The optical properties of the maternal abdominal tissue 90 and the amniotic fluid 94, and the position of the maternal abdominal tissue and amniotic fluid in the optical path, are largely unchanged when the light emitter and detector are fixed to the maternal abdomen and the mother stays still during monitoring. This means that any significant variation in the intensity and pattern of received light are primarily caused by fetal movement.

When the fetus 92 stays still, whether the fetus is in the optical path or not, the constitution of the propagation paths, for example the proportion of the maternal tissues 90, the fetal tissues 92 and the amniotic fluid 94, keeps relatively constant, which results in a stable influence over the light travelling through. Then the received light at the light receiver 12 has a constant intensity and pattern. Once the fetus moves, either away from or into the optical path as represented by arrow 96 in FIG. 9(b), the intensity and pattern of received light changes as the composition of the light propagation channel changes, which are caused by the disturbance of the fetus.

Thus, this second approach is based on monitoring the optical path disturbance caused when the fetus moves. In this way, the system can measure not only the fetal movements which result in touching of the maternal abdomen but also fetal movements within the womb without touching maternal abdomen.

This approach requires light which penetrates to a depth such that the optical path is affected by the fetus. Red and infrared light (radiation) are preferably used rather than green light. The absorption of blood in the green spectrum region is so large that green light can only be transmitted through very thin layers of tissue, whereas red and infrared light can reach a high penetration depth to reach the fetus.

As in the approach above, signal processing may be used to eliminate potential influencing factors on the received signals and differentiate between different movement patterns.

For example, the direct current signal from the sensor may first be filtered out using a high pass filter (or band pass filter). In this way, when the fetus is stationary, no matter if it is blocking the optical path or not, the output is zero.

Signal processing is then used to differentiate different types of fetal movement from the received light intensity and pattern. For example, it may be possible to distinguish between fetal movements which touch the maternal abdomen and fetal movements within the womb which do not touch the maternal abdomen. External influencing factors, such as maternal movement or external forces which generate pressure on the maternal abdomen, may also be cancelled using motion sensing as described above, and by suitable design of the belt.

In order to distinguish between different movement patterns, one example makes use of four features:

(i) the highest peak value in the time domain;

(ii) the lowest valley value in the time domain (i.e. the valley with the greatest magnitude);

(iii) the ratio of the second highest peak value to second lowest valley value (i.e. the valley with the second greatest magnitude) in the time domain;

(iv) the frequency of the highest peak in the frequency domain.

The implementation of the system is as shown in FIGS. 4 and 5. The controller 30 is used to analyze both intensity changes and pattern changes, in order to identify fetal movements.

In addition, by analyzing the received light signal, maternal physiological information including heart rate, heart rate variability and oxygen saturation can be generated as explained above, but also fetal physiological information, again including heart rate, heart rate variability and oxygen saturation. The signal processing is thus able to distinguish between irregular variations in the received light intensity and patterns, which indicate fetal movement, and the regular variations which are mainly caused by maternal respiration and cardiac pulse.

Experiments have also been conducted to demonstrate the effectiveness of the approach, by examining how PPG signals (such as using pulse oximetry) change in response to internal disturbances of the light path.

A simulated maternal abdomen (2 cm in thickness) and fetus were employed. An LED emitter and photodetector were attached to the simulated maternal abdomen with a separation distance of around 2 cm.

Artificial fetal movements and potential influencing factors which may induce optical signal disturbance were created. In total, three patterns of disturbance were analyzed experimentally:

(i) A touch (T) event, by using fingers to tap or press the surface of the belly mimicking maternal movement or other influencing factors which generate pressure on the maternal abdomen;

(ii) A kick & touch (KT) event, in which the fetus kicks and touches the belly;

(iii) A kick (K) event, in which the fetus kicks without touching the belly.

A periodic oscillating noise with 50 Hz frequency was added to the collected data sets to simulate electrical noise.

FIG. 10 shows in the top plot a raw collected PPG signal (with the DC component removed), and in the bottom plot it shows the signal after low pass filtering to remove the 50 Hz noise. It shows the PPG signal response to a tap on the belly. The removal of the DC component and the filtering of the noise may together be achieved with a band pass filter.

FIG. 11 shows the PPG signal (versus time) recorded in response to successive tapping events, based on a repetition time of 1 second.

It can be seen that each tapping event generates a pulse with near equal positive magnitude peak and negative magnitude valley, and there are few subsequent oscillations.

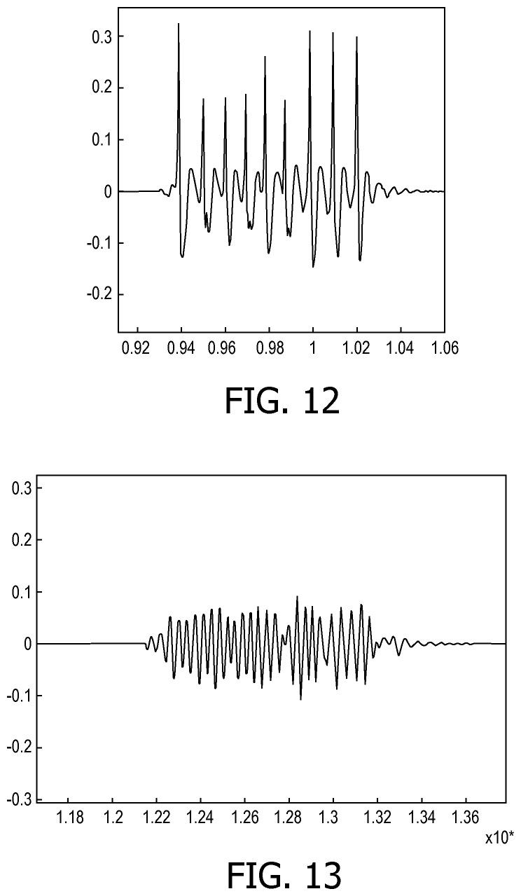

FIG. 12 shows the PPG signal (versus time) recorded in response to successive kick and tap events, based on a repetition time of 1 second.

It can be seen that each event generates a pulse with a larger positive magnitude peak than the deepest negative magnitude valley, but again there are few subsequent oscillations.

FIG. 13 shows the PPG signal (versus time) recorded in response to successive kick events, again based on a repetition time of 1 second.

It can be seen that each event generates an oscillating response roughly centered around zero.

FIGS. 11 to 13 thus each show successive examples of only one event type (T, KT or K).

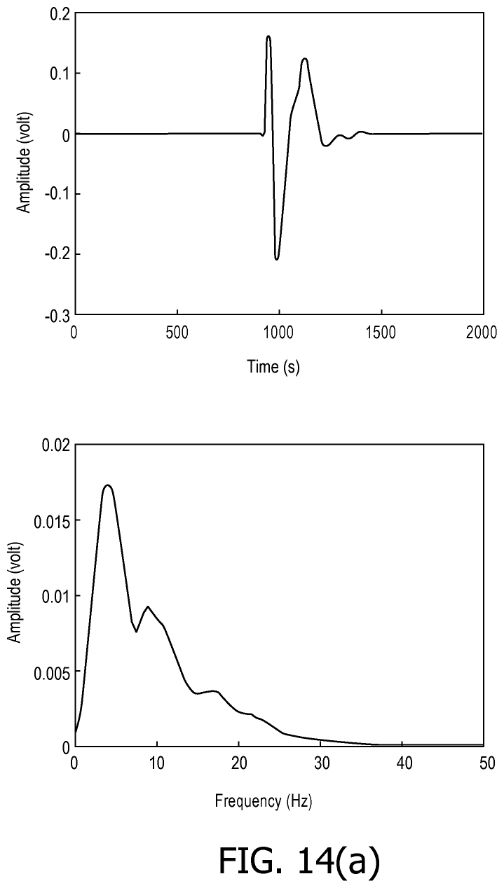

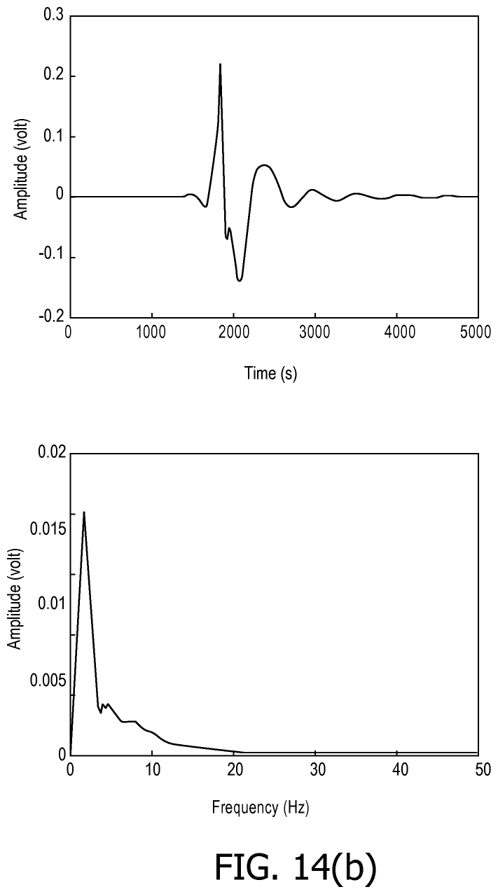

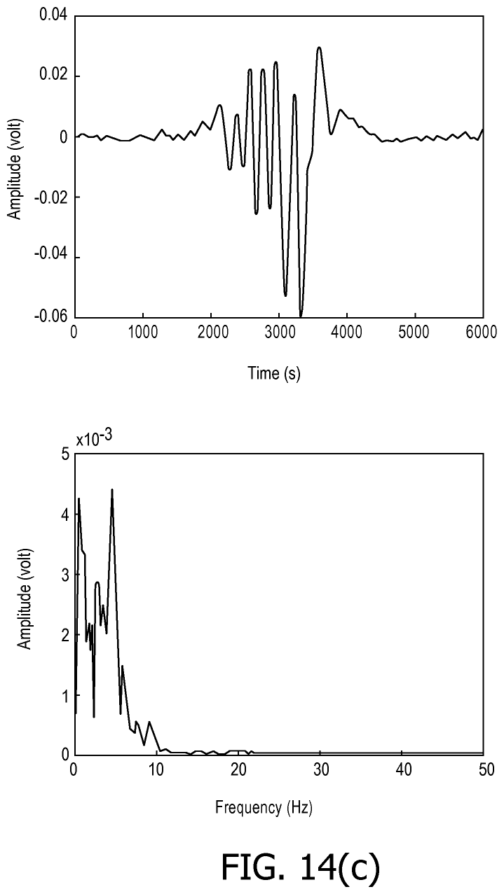

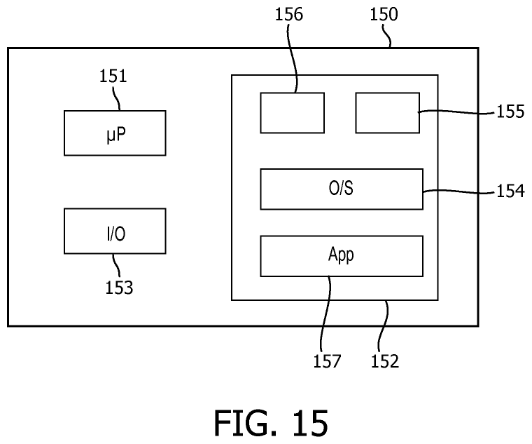

FIG. 14 shows one individual event of each type, both in the time domain and frequency domain. FIG. 14(a) shows a tap event (T), FIG. 14(b) shows a tap and kick event (KT) and FIG. 14(c) shows a kick event (K). The top plots are the time domain plots and the bottom plots are frequency domain plots.

In the frequency domain, the kick event generates multiple frequency components. The tap and kick event has the lowest main frequency component.

These differences in time domain and frequency domain signal pattern may be used to distinguish between different types of event.

For each event, the following features in both time and frequency domains may for example be extracted:

the peaks and valleys in the time domain with the top 2 amplitudes and their differences and ratio;

the peaks in the frequency domain with the top 2 amplitudes and their difference and ratio.

In the experiments, recordings were made of 68 tap events, 68 tap and kick events and 55 kick events.

The time domain and frequency domain features were then substituted into a decision tree classifier with a cross validation of 10 folds. Four of the features were used in the classification. These four features were:

(i) the highest peak value in the time domain;
(ii) the lowest valley value in the time domain;
(iii) the ratio of the second highest peak value to second lowest valley value in time domain; and
(iv) the frequency of the highest peak in the frequency domain.

Feature (i) is of particular interest in identifying the kick and tap event, which generally has a highest peak value (see FIG. 12).

Feature (ii) is of particular interest in identifying the tap event, which generally has a largest magnitude valley value relative to the highest peak value (see FIG. 11).

Feature (iii) is of particular interest in identifying the kick event, which generally has an un-damped response so that the second highest peak and second deepest valley are still relatively large.

Feature (iv) is of particular interest in identifying the kick and tap event, which generally has the lowest main frequency component (see FIG. 14(b)).

There are of course other metrics which may be used.

In general, one or more parameters which characterize the signals are obtained in the time domain and/or the frequency domain.

The results of this particular approach are shown in Table 1 below. Each column shows how many events were classified in each of the three categories.

| T | KT | T |
|---|---|---|
| 64 | 3 | 1 |
| 3 | 65 | 0 |
| 3 | 2 | 50 |

In this experiment 93.7% (179 out of 191) instances were correctly classified. A large range of strengths of movements was also used. For example, in the tap events, movements giving rise to sensed signal amplitude ranging from 0.1 to 0.5 volt were recorded. This shows that the algorithm for identifying the movement pattern is not very sensitive to the absolute amplitude of the movement.

The system is thus able to count the fetal movements, as well as distinguishing between movements in which the fetus has touched the belly, and exclude the false alarms caused by maternal movements.

The system described above makes use of a controller or processor for processing the sensed data and for performing the data analysis.

FIG. 15 illustrates an example of a computer 150 for implementing the controller or processor described above.

The computer 150 includes, but is not limited to, PCs, workstations, laptops, PDAs, palm devices, servers, storages, and the like. Generally, in terms of hardware architecture, the computer 150 may include one or more processors 151, memory 152, and one or more I/O devices 153 that are communicatively coupled via a local interface (not shown). The local interface can be, for example but not limited to, one or more buses or other wired or wireless connections, as is known in the art. The local interface may have additional elements, such as controllers, buffers (caches), drivers, repeaters, and receivers, to enable communications. Further, the local interface may include address, control, and/or data connections to enable appropriate communications among the aforementioned components.

The processor 151 is a hardware device for executing software that can be stored in the memory 152. The processor 151 can be virtually any custom made or commercially available processor, a central processing unit (CPU), a digital signal processor (DSP), or an auxiliary processor among several processors associated with the computer 150, and the processor 151 may be a semiconductor based microprocessor (in the form of a microchip) or a microprocessor.

The memory 152 can include any one or combination of volatile memory elements (e.g., random access memory (RAM), such as dynamic random access memory (DRAM), static random access memory (SRAM), etc.) and non-volatile memory elements (e.g., ROM, erasable programmable read only memory (EPROM), electronically erasable programmable read only memory (EEPROM), programmable read only memory (PROM), tape, compact disc read only memory (CD-ROM), disk, diskette, cartridge, cassette or the like, etc.). Moreover, the memory 152 may incorporate electronic, magnetic, optical, and/or other types of storage media. Note that the memory 152 can have a distributed architecture, where various components are situated remote from one another, but can be accessed by the processor 151.

The software in the memory 152 may include one or more separate programs, each of which comprises an ordered listing of executable instructions for implementing logical functions. The software in the memory 152 includes a suitable operating system (O/S) 154, compiler 155, source code 156, and one or more applications 157 in accordance with exemplary embodiments.

The application 157 comprises numerous functional components such as computational units, logic, functional units, processes, operations, virtual entities, and/or modules.

The operating system 154 controls the execution of computer programs, and provides scheduling, input-output control, file and data management, memory management, and communication control and related services.

Application 157 may be a source program, executable program (object code), script, or any other entity comprising a set of instructions to be performed. When a source program, then the program is usually translated via a compiler (such as the compiler 155), assembler, interpreter, or the like, which may or may not be included within the memory 152, so as to operate properly in connection with the operating system 154. Furthermore, the application 157 can be written as an object oriented programming language, which has classes of data and methods, or a procedure programming language, which has routines, subroutines, and/or functions, for example but not limited to, C, C++, C#, Pascal, BASIC, API calls, HTML, XHTML, XML, ASP scripts, JavaScript, FORTRAN, COBOL, Perl, Java, ADA, .NET, and the like.

The I/O devices 153 may include input devices such as, for example but not limited to, a mouse, keyboard, scanner, microphone, camera, etc. Furthermore, the I/O devices 153 may also include output devices, for example but not limited to a printer, display, etc. Finally, the I/O devices 153 may further include devices that communicate with both inputs and outputs, for instance but not limited to, a network interface controller (NIC) or modulator/demodulator (for accessing remote devices, other files, devices, systems, or a network), a radio frequency (RF) or other transceiver, a telephonic interface, a bridge, a router, etc. The I/O devices 153 also include components for communicating over various networks, such as the Internet or intranet.

When the computer 150 is in operation, the processor 151 is configured to execute software stored within the memory 152, to communicate data to and from the memory 152, and to generally control operations of the computer 150 pursuant to the software. The application 157 and the operating system 154 are read, in whole or in part, by the processor 151, perhaps buffered within the processor 151, and then executed.

When the application 157 is implemented in software it should be noted that the application 157 can be stored on virtually any computer readable medium for use by or in connection with any computer related system or method. In the context of this document, a computer readable medium may be an electronic, magnetic, optical, or other physical device or means that can contain or store a computer program for use by or in connection with a computer related system or method.

The system and method described above may be used for fetal/pregnancy monitoring products.

The examples above make use of a PPG sensor. This may be implemented by a pulse oximetry sensor for example. Other optical emitter and sensor arrangements may also be used.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

1. A fetal movement monitoring system comprising:
an abdominal belt;
an optical sensor arrangement carried by the belt, comprising at least one light emitter for emitting an optical signal toward abdomen of an object and at least one light receiver for receiving the optical signal from the abdomen of the object and
a controller adapted to analyze optical signals received by the light receiver of the optical sensor arrangement thereby to detect fetal movements based on a pattern change of the received optical signal, wherein the optical signal is directed to travel through the abdomen of the object.

2. A system as claimed in claim 1, wherein the optical sensor arrangement comprises at least one PPG sensor.

3. A system as claimed in claim 2, wherein the optical sensor arrangement comprises at least one pulse oximetry sensor.

4. A system as claimed in claim 1, comprising a memory for storing fetal movement information.

5. A system as claimed in claim 1, further comprising a motion sensor.

6. A system as claimed in claim 5, wherein the motion sensor is carried by the belt, away from the fetal area.

7. A system as claimed in claim 5, wherein the motion sensor, or a further motion sensor, is worn by the expectant mother away from the belt.

8. A system as claimed in claim 1, wherein the pattern of the optical signals received is characterized by at least one time domain parameter or at least one frequency domain parameter or the combination of both.

9. A system as claimed in claim 1, further comprising an output device, wherein the controller or the optical sensor arrangement communicates wirelessly with the output device.

10. A system as claimed in claim 9, wherein the output device comprises a watch or a phone.

11. A system as claimed in claim 1, wherein the light emitter is adapted to emit:
green light; or
red light or infrared radiation.

12. A fetal movement monitoring method comprising:
emitting an optical signal by at least one light emitter toward a fetal area of an object and receiving the optical signal from the fetal area the object by at least one light receiver using an optical sensor arrangement comprising the at least one light emitter and the at least one light receiver provided on an abdominal belt;
analyzing the optical signal received by the light receiver thereby to detect a fetal movement based on a pattern change of the received optical signal,
wherein the optical signal is directed to travel through the abdomen of the object.

13. A method as claimed in claim 12, comprising using a PPG sensor to generate and receive the optical signal.

14. A method as claimed in claim 12, further comprising using motion information relating to the expectant mother to assist in analyzing the optical sensor signals.

15. A system as claimed in claim 1, comprising generating an optical signal with:
green light; or
red light or infrared radiation.

16. A method as claimed in claim 12, further comprising communicating a detected fetal movement to an output device.

17. A method as claimed in claim 12, further comprising measuring a change in the optical path between the optical sensor and detector.

18. A method as claimed in claim 12, further comprising measuring a difference in contact pressure with the optical sensor arrangement.

19. A system as claimed in claim 1, wherein the controller is further adapted to measure a change in the optical path between the optical sensor and detector.

20. A system as claimed in claim 1, wherein the controller is further adapted to measure a difference in contact pressure with the optical sensor arrangement.
